(19) United States
(12) Patent Application Publication
(10) Pub. No.: US 2026/0240555 A1
(43) Pub. Date: Aug. 20, 2026

(54) SHOCKWAVE BALLOON DEVICES

(71) Applicant: JIANGSU REYNOLDS MEDICAL TECHNOLOGY CO., LTD., Nantong (CN)

(72) Inventors: Xinpeng XU, Nantong (CN); Haixia ZHAO, Nantong (CN)

(73) Assignee: JIANGSU REYNOLDS MEDICAL TECHNOLOGY CO., LTD., Nantong (CN)

(21) Appl. No.: 19/231,544

(22) Filed: Jun. 8, 2025

Related U.S. Application Data
(63) Continuation-in-part of application No. PCT/CN2025/088716, filed on Apr. 14, 2025.

(30) Foreign Application Priority Data
Feb. 17, 2025 (CN) 202510173219.X

(51) Int. Cl. A61B 17/22; A61M 25/10

(52) U.S. Cl. CPC A61B 17/22022; A61B 2017/22025; A61B 2017/22062; A61B 2017/22084; A61B 2017/22092; A61B 2017/22098; A61M 25/1002; A61M 2025/109

(57) ABSTRACT

A shockwave balloon device is provided. The shockwave balloon device comprises a handle structure and a balloon structure connected with the handle structure; the handle structure includes a handle and an outer tube, the outer tube is fixedly disposed at a distal end of the handle, the handle is slidably disposed with a sliding button, and the sliding button is fixedly connected with a proximal end of the balloon structure; the balloon structure includes an intermediate tube, an inner tube, a balloon, and a tip tube, the intermediate tube is disposed on an outer side of the inner tube, the balloon is disposed on the outer side of the inner tube, and a proximal end of the intermediate tube is fixedly connected with the sliding button.

### CROSS-REFERENCE TO RELATED APPLICATIONS

This application is a continuation-in-part of International Application No. PCT/CN2025/088716, filed on Apr. 14, 2025, which claims priority to Chinese Patent Application No. 202510173219.X, filed on Feb. 17, 2025, the entire contents of each of which are hereby incorporated by reference.

### TECHNICAL FIELD

The present disclosure relates to the field of medical devices, and in particular to a shockwave balloon device.

### BACKGROUND

Calcific aortic valve disease (CAVD), as a common valvular heart disease, has a spectrum of conditions ranging from aortic valve sclerosis to calcific aortic stenosis (CAS). The aortic valve sclerosis is often a precursor to aortic stenosis, with approximately 9% of cases progressing to aortic stenosis within 5 years; the cardiovascular health study found that approximately 1%-2% of aortic valve sclerosis cases progress to aortic stenosis each year. 75% of patients who have already developed aortic stenosis require valve replacement surgery due to heart failure or may die within 2 to 5 years. In the clinical pathogenesis, a stiffness of the valve leaflet of calcific aortic stenosis progressively increases, and advanced lesions resemble bone formation, with calcification as a prominent feature. Currently, transcatheter aortic valve implantation (TAVI) is a main treatment for the calcific aortic stenosis, while pharmacologic therapy is still under investigation.

Although prosthetic valves have been shown to be safe and effective in clinical use, the prosthetic valves are costly to implant, and European System for Cardiac Operative Risk Evaluation (Euro SCORE) evaluates that the mortality risk is greater than 20% and the Society of Thoracic Surgeons mortality risk score (STS) evaluates that the mortality risk is greater than 10%. Additionally, prosthetic valves recalcify and fail over time after implantation. With the growing emphasis on the “intervention without implantation” concept, both patients and clinicians increasingly hope for a low-risk, non-implantable device that can soften the valve and delay the need for prosthetic valve implantation, rather than immediately proceeding with valve replacement surgery, while also aiming to improve the function of calcified prosthetic valves.

In recent years, endovascular shockwave technology has evolved. In the field of intracoronary balloon shockwave therapy, clinical research data published by numerous international companies have fully confirmed the safety and efficacy of intravascular shockwave treatment for calcification. In the field of heart valves, many international companies and research institutions have conducted in-depth studies on the effects of ultrasonic cavitation and shockwave cavitation in treating valvular calcification. The results have shown that both ultrasonic cavitation and shockwave cavitation can effectively soften calcified valves and biological tissues.

Based on this, focusing on the application of shockwave cavitation, it is desirable to provide a safe, convenient, and highly effective device to meet the urgent need for low-risk, non-implantable treatment solutions in the current field of cardiovascular disease treatment.

### SUMMARY

One or more embodiments of the present disclosure provide a shockwave balloon device. The shockwave balloon device comprises: a handle structure and a balloon structure connected with the handle structure; wherein the handle structure includes a handle and an outer tube, the outer tube is fixedly disposed at a distal end of the handle, the handle is slidably disposed with a sliding button, and the sliding button is fixedly connected with a proximal end of the balloon structure; the balloon structure includes an intermediate tube, an inner tube, a balloon, and a tip tube, the intermediate tube is disposed on an outer side of the inner tube, the balloon is disposed on the outer side of the inner tube, a proximal end of the intermediate tube is fixedly connected with the sliding button, a distal end of the intermediate tube is fixedly connected with the balloon, and the tip tube is fixedly disposed at a distal end of the balloon; and the intermediate tube is slidably coupled with the outer tube.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating a structure of a shockwave balloon device according to some embodiments of the present disclosure;

FIG. 2 is a schematic diagram illustrating a structure of a circular-arc surface of a balloon according to some embodiments of the present disclosure;

FIG. 3 is a schematic diagram illustrating a cross-sectional structure of an outer tube, an intermediate tube, and an inner tube according to some embodiments of the present disclosure;

FIG. 4 is a schematic structural illustrating a cross-sectional structure of an inner tube according to some embodiments of the present disclosure;

FIG. 5 is an enlarged view of region A in FIG. 1;

FIG. 6 is a schematic diagram illustrating a structure of electrode assemblies according to some embodiments of the present disclosure;

FIG. 7A is a schematic diagram illustrating a structure of a clamping arm device according to some embodiments of the present disclosure; FIG. 7B is a schematic diagram illustrating another structure of a clamping arm device according to some embodiments of the present disclosure.

### DETAILED DESCRIPTION

In the following detailed description, numerous specific details are set forth by way of examples in order to provide a thorough understanding of the relevant disclosure. Obviously, drawings described below are only some examples or embodiments of the present disclosure. Those skilled in the art, without further creative efforts, may apply the present disclosure to other similar scenarios according to these drawings. It should be understood that the purposes of these illustrated embodiments are only provided to those skilled in the art to practice the application, and not intended to limit the scope of the present disclosure. Unless obviously obtained from the context or the context illustrates otherwise, the same numeral in the drawings refers to the same structure or operation.

It will be understood that the terms “system,” “engine,” “unit,” “module,” and/or “block” used herein are one method to distinguish different components, elements, parts, sections, or assemblies of different levels in ascending order. However, the terms may be displaced by other expressions if they achieve the same purpose.

The terminology used herein is for the purposes of describing particular examples and embodiments only and is not intended to be limiting. As used herein, the singular forms “a,” “an,” and “the” may be intended to include the plural forms as well, unless the context clearly indicates otherwise. It will be further understood that the terms “include” and/or “comprise,” when used in this disclosure, specify the presence of integers, devices, behaviors, stated features, steps, elements, operations, and/or components, but do not exclude the presence or addition of one or more other integers, devices, behaviors, features, steps, elements, operations, components, and/or groups thereof.

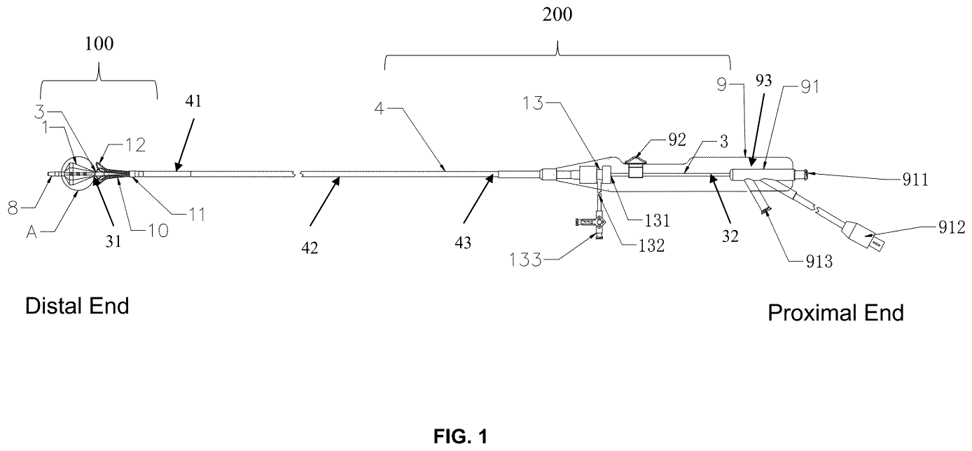

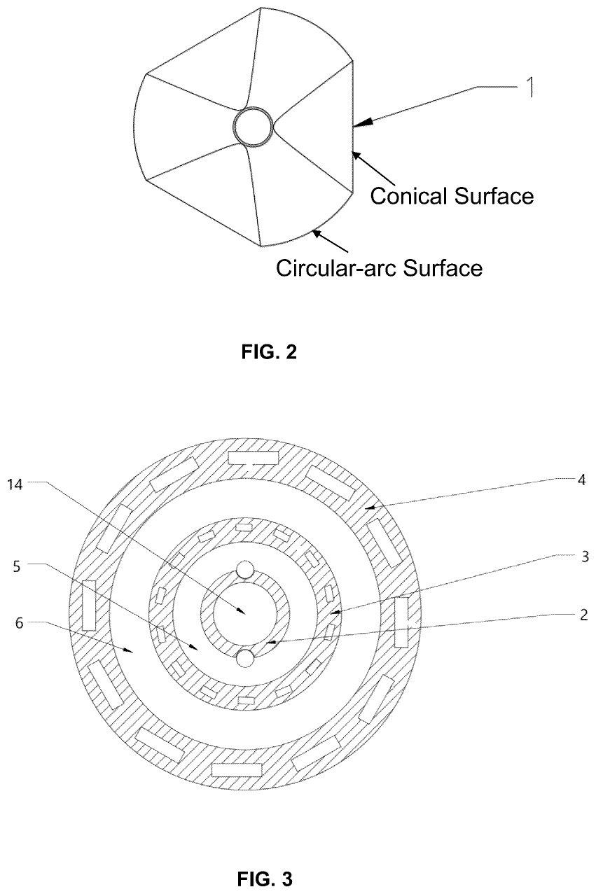

FIG. 1 is a schematic diagram illustrating a structure of a shockwave balloon device according to some embodiments of the present disclosure. FIG. 2 is a schematic diagram illustrating a structure of a circular-arc surface of a balloon according to some embodiments of the present disclosure. FIG. 3 is a schematic diagram illustrating a cross-sectional structure of an outer tube, an intermediate tube, and an inner tube according to some embodiments of the present disclosure.

As shown in FIG. 1 to FIG. 3, some embodiments of the present disclosure provide a shockwave balloon device, the shockwave balloon device includes a handle structure 200 and a balloon structure 100 connected with the handle structure 200.

The handle structure 200 refers to a structure that is convenient for an operator to hold. The operator includes a physician, a nurse, or the like.

The balloon structure 100 is a structure in the shockwave balloon device that is capable of intervening at a target location (e.g., a valve). In some embodiments, the operator is able to control the balloon structure 100 to navigate along the vascular pathway until reaching the lesion site in the coronary artery.

In some embodiments, the handle structure 200 includes a handle 9 and an outer tube 4, the outer tube 4 is fixedly disposed at a distal end of the handle 9, the handle 9 is slidably disposed with a sliding button 92, and the sliding button 92 is fixedly connected with a proximal end of the balloon structure 100.

It should be noted that “the proximal end” and “the distal end” in some embodiments of the present disclosure may be defined with reference to the operator using the shockwave balloon device; an end toward the operator is “the proximal end” and an end away from the operator is “the distal end”. In some embodiments, “the proximal end” and “the distal end” may denote an endpoint, an end face, an end portion, and a portion near the end portion having a certain length of a member of the shockwave balloon device. For example, an end proximate to the balloon structure that is inserted into the human body is defined as “the distal end”; the other end (i.e., an endpoint, an end face, an end portion, or a portion near the end portion having a certain length, which is located opposite the distal end of the shockwave balloon device, is defined as “the proximal end”.

There are a plurality of ways of fixedly setting or fixedly connection. For example, a portion of the outer tube 4 near the handle 9 is fixedly connected with the handle 9 by integrally molded, glued, welded, or the like. There are a plurality of ways of slidably setting or slidingly coupling. For example, the sliding button 92 is provided at a groove on the main body 93 of the handle 9, which is capable of sliding relative to the groove. As another example, the sliding button 92 is a clearance fit with the handle 9 to be able to slide relatively. The handle 9 provided at the proximal end can be easily gripped and operated by the physician.

In some embodiments, the balloon structure 100 includes: an intermediate tube 3, an inner tube 2, a balloon 1, and a tip tube 8. The intermediate tube 3 is sheathed on an outer side of the inner tube 2, and the balloon 1 is sheathed on the outer side of the inner tube 2. A proximal end of the intermediate tube 3 is fixedly connected with the sliding button 92, and a distal end of the intermediate tube 3 is fixedly connected with the balloon 1. The tip tube 2 is fixedly disposed at a distal end of the balloon 1. The intermediate tube 3 is slidably coupled with the outer tube 4. In some embodiments, the intermediate tube 3 is coaxially sheathed on the outer side of the inner tube 2, and the outer tube 4 is coaxially sheathed on the outer side of the intermediate tube 3; the proximal end of the inner tube 2 penetrates the balloon 1. In other words, an axis of the balloon 1, an axis of the inner tube 2, and an axis of the intermediate tube 3 are the same.

The balloon 1 may be made of soft and flexible materials, and the balloon 1 is able to expand under pressure, thereby helping to dilate narrowed or blocked blood vessels or tissues. For example, an interior of the balloon 1 has a cavity, and the cavity may be filled with a contrast agent, and the more the contrast agent there is, the more the balloon 1 expands, which exerts pressure on the calcified valve, prompting a calcified fracture of the valve, thereby softening the valve.

In some embodiments, materials of the balloon 1 include nylon and nylon derivatives. The balloon 1 is made of nylon or nylon derivatives, since the acoustic impedance of this materials is highly matched with that of the internal liquid after balloon pressurization, the impedance matching can effectively reduce ultrasonic reflection losses at the interface, ensure that ultrasonic energy is evenly distributed, thereby improving the efficiency and accuracy of the shockwave treatment process, and ultimately achieving an optimal ultrasonic energy transfer effect.

As shown in FIG. 3, there are the inner tube 2, the intermediate tube 3, and the outer tube 4 in order from an inner side to an outer side. As shown in FIG. 1, the balloon 1 is sheathed on the outer side of the distal end of the inner tube 2, and the intermediate tube 3 is sheathed on the outer side of the proximal end of the inner tube 2. I.e., circumferentially, the distal end of the intermediate tube 3 is at a same level as the balloon 1. From the distal end to the proximal end, there are provided with the tip tube 8, the balloon 1, the outer tube 4, and the sliding button 92 in order.

When the operator pushes the sliding button 92, the sliding button 92 drives the intermediate tube 3 to slide relative to the outer tube 4. The movement of the intermediate tube 3 drives the inner tube 2, the balloon 1, and the tip tube 8 to slide, and the balloon 1 is pushed into the target position (e.g., below the valve).

In some embodiments, a distal end of the tip tube 8 is a chamfered opening with a tapered profile. The chamfered opening of the tip tube 8 can facilitate the puncturing of the balloon structure into the blood vessel.

In some embodiments, the shockwave balloon device further includes electrode assemblies, a clamping arm device, and a hemostatic valve. More descriptions regarding the electrode assemblies, the clamping arm device, and the hemostatic valve may be found in the related descriptions below.

In some embodiments, the shockwave balloon device is used as follows. The physician holds and operates the sliding button 92 on the handle 9, which is fixedly connected with the proximal end of the intermediate tube 3. By operating the sliding button 92, the intermediate tube 3 is driven forward, thereby pushing the balloon 1 and enabling precise control of movement of the balloon structure. Meanwhile, the physician may also utilize X-ray to closely observe the marking of the balloon 1 so as to precisely push the balloon 1 under the valve.

In some embodiments of the present disclosure provide the shockwave balloon device, since the shockwave balloon device is low-risk and non-implantable, the shockwave balloon device is able to meet the urgent need for non-implantable treatment solutions in the current field of cardiovascular disease treatment.

In some embodiments, as shown in FIG. 3, a first gap is arranged between the inner tube 2 and the intermediate tube 3 to form a first channel 5, the first channel 5 is in communication with a cavity within the balloon 1; and a second gap is arranged between the intermediate tube 3 and the outer tube 4 to form a second channel.

The first channel 5 may be configured to deliver a contrast agent. For example, the contrast agent in the balloon 1 may flow into or out of the balloon 1 through the first channel 5. The second channel 6 may be configured to deliver in or out other desired media. For example, the second channel 6 may be configured to deliver the contrast agent in or out. As another example, the second channel 6 may be configured to deliver heparin, the anticoagulant effect of the heparin can reduce the formation of blood clots. In some embodiments, the second outlet channel 132 is in communication with the second channel 6 and a three-way valve 133 of the second outlet channel 132, facilitating the three-way valve 133 for delivering the contrast agent or heparin.

In some embodiments of the present disclosure, the shockwave balloon device does not require introduction of an additional contrast catheter during the treatment process, as the contrast agent channel is provided above the valve through the first channel and below the valve through the second channel; the reduction of the contrast catheter can reduce the trauma of the device to the patient. Furthermore, the increase of the count of contrast agent channels improves visualization for the physician during the procedure.

In some embodiments, as shown in FIG. 1, the handle 9 includes a main body 93 and a connector 91. The connector 91 includes a third channel 911, a fourth channel 912, and a fifth channel 913. The third channel 911, the fourth channel 912, and the fifth channel 913 penetrate the main body 93. The third channel 911 is in communication with a tube lumen 14 of the inner tube 2, the fourth channel 912 is provided for passage of the positive electrode wire 71 and the negative electrode wire 72, and the fifth channel 913 is in communication with the first channel 5. The third channel 911 may serve as a channel for guidewires. For example, the third channel 911 may accommodate a vascular interventional guidewire with a 0.035-inch size.

The third channel 911, the fourth channel 912, and the fifth channel 913 may be formed from an internal cavity within the main body 93. The third channel 911, the fourth channel 912, and the fifth channel 913 may not be in communication.

The positive electrode wire 71 and the negative electrode wire 72 may be connected with the electrode assemblies (e.g., as shown in FIG. 5, the first electrode set 21, the second electrode set 22 or the third electrode set 23) disposed inside the inner tube 2 to provide power to the electrode assemblies for applying a shock wave to the calcified valve after intervention of the valve by the shockwave balloon device.

In some embodiments, the positive electrode wire 71 and the negative electrode wire 72 may be connected with a high-voltage plug.

In some embodiments, as shown in FIG. 1, the outer tube is a connecting section 41, a soft section 42, and a pushing section 43 in order from the distal end to the proximal end.

In some embodiments of the present disclosure, the connecting section 41 is configured to connect the clamping arm device 10. The relatively high stiffness of the pushing section 43 facilitates pushing, and the moderate softness and specific length of the soft section 42 facilitate bending, which can allow the shockwave balloon device to be smoothly pushed and effectively adapt to the curvature and softness of the blood vessel inside the vessel, thereby reducing damage to the blood vessel wall.

In some embodiments, the soft section 42 and the pushing section 43 are made of braided filaments. Materials of the braided filaments may include stainless steel wires, nickel-titanium alloy wires, polymer reinforced fibers, or the like. The process of braiding may include cross or bi-directional braiding, multi-layer composite braiding, or the like. In some embodiments, the soft section 42 and the pushing sections 43 are braided with the nickel-titanium alloy wires.

In some embodiments, a hardness of the pushing section 43 is greater than a hardness of the soft section 42, the hardness of the soft section 42 is in a range of 35D~55D, and a length of the soft section 42 is in a range of 10 cm~35 cm. For example, the hardness of the soft section 42 is 40D, and the hardness of the pushing section is 60D; the length of the soft section 42 is 20 cm. As another example, the hardness of the soft section 42 is 35D, and the hardness of the pushing section 43 is 40D; the length of the soft section 42 is 10 cm. As a further example, the hardness of the soft section 42 is 50D, and the hardness of the pushing section 43 is 60D; the length of the soft section 42 is 35 cm.

In some embodiments, an inner wall of the outer tube is coated with a Polytetrafluoroethylene (PTFE) coating or lubricant. The lubricant may include polyvinylpyrrolidone, polyacrylic acid, and hydrophobic polymers such as PTFE.

In some embodiments of the present disclosure, the inner wall of the outer tube is coated with the PTFE coating or lubricant, which reduces the friction of the sliding of the intermediate tube with respect to the outer tube when the intermediate tube is driven by the sliding button to slide. When the contrast agent is injected, the PTFE coating or lubricant can reduce the shear force between the contrast agent and the tubing and increase the flow rate of the contrast agent. If blood enters a lumen, the PTFE coating or lubricant facilitates fluid flushing and prevents blood from coagulating and adhering to the lumen.

In some embodiments, a hemostatic valve 13 is fixedly provided at a proximal end of the outer tube 4; the handle 9 includes a main body 93; an interior of the main body 93 has a cavity and the hemostatic valve 13 is fixedly disposed in the main body 93.

The hemostatic valve 13 refers to a valve that is configured to prevent a backflow of blood or other body fluids through an inlet of the device. During the procedure, the hemostatic valve 13 can serve to prevent blood from spilling out, maintain a sterile operating environment, and maintain system pressure.

In some embodiments, the hemostatic valve 13 includes a first outlet channel 131 and a second outlet channel 132. A silicone sealing ring is fixedly provided in the first outlet channel 131, the proximal end of the intermediate tube 3 passes through the silicone sealing ring, and the silicone sealing ring is configured to seal the second channel 6. The second outlet channel 132 is in communication with the second channel 6, a side of the second outlet channel 132 away from the outer tube 4 extends beyond a side wall of the main body 93, and the second outlet channel 132 is fixedly connected with a three-way valve 133.

The silicone sealing ring can prevent blood, body fluids, or contrast agent from flowing out of the second channel 6. The three-way valve 133 may be configured to deliver the contrast agent or heparin. For example, the three-way valve 133 is coupled with the second outlet channel 132 and the second channel 6 to deliver contrast agent or heparin into the second channel 6. The silicone sealing ring may be disposed at a position close to the proximal end relative to the three-way valve 133.

In some embodiments, a compressive strength range of the silicone sealing ring is 200 psi to 500 psi. For example, the compressive strength of the silicone sealing ring is 200 psi. As another example, the compressive strength of the silicone sealing ring is 400 psi. As a further example, the compressive strength of the silicone sealing ring is 250 psi.

In some embodiments of the present disclosure, by the provision of the hemostatic valve, the three-way valve, it is convenient for the operator to deliver the contrast agent or heparin to a channel within the shockwave balloon device, which increases functions of the shockwave balloon device.

In some embodiments, the intermediate tube 3 includes a distal section 31 and a proximal section 32. The distal section 31 of the intermediate tube 3 is made of a braided tube constructed from interwoven filaments, and a hardness of the distal section 31 of the intermediate tube 3 is in a range of 50D to 70D. The proximal section 32 of the intermediate tube 3 is a metal tube, and a length of the proximal section 32 is greater than a distance from the connector 91 to the silicone sealing ring at the three-way valve 133. For example, the hardness of the distal section 31 of the intermediate tube 3 is 60D, and the proximal section 32 of the intermediate tube 3 is a metallic tube made of stainless steel; the proximal section 32 of the intermediate tube 3 extends from the connector 91 to the silicone sealing ring of the three-way valve 133. As another example, the hardness of the distal section 31 of the intermediate tube 3 is 50D, and the proximal section 32 of the intermediate tube 3 is a metal tube made of the nickel-titanium alloy; the proximal section 32 of the intermediate tube 3 extends from the connector 91 to a position of the pushing section 43 as shown in FIG. 1. As another example, the hardness of the distal section 31 of the intermediate tube 3 is 70D, and the proximal section 32 of the intermediate tube 3 is a metal tube made of the nickel-titanium alloy; the proximal section 32 of the intermediate tube 3 extends from the connector 91 to a farthest end of the main body 93 of the handle 9.

A process of braiding the intermediate tube 3 and the outer tube 4 and the materials of the braided filaments may be the same or different. More descriptions regarding the process of braiding the braided filaments and the materials of the braided filaments may be found in the related descriptions above.

By designing the materials, hardness, and size of the different sections of the intermediate tube 3, it is possible to simultaneously satisfy the smooth pushing and the sealing performance of the silicone sealing ring at the three-way valve 133, thus providing the reliability of the shockwave balloon device.

In some embodiments, the handle 9 includes a main body 93 and a connector 91. A proximal end of the connector 91 penetrates a proximal end of the main body 93, and the connector 91 is slidably disposed at the proximal end of the main body 93. A groove is provided in a side of the main body 93, the sliding button 92 is slidably disposed in the groove, and a side of the sliding button 92 is fixedly connected with an outer wall of a proximal end of the intermediate tube 3; and the proximal end of the intermediate tube 3 and a proximal end of the inner tube 2 are fixedly connected with a distal end of the connector 91.

In some embodiments, the connector 91 is provided inside the proximal end of the main body 93 and passes through an interior of the proximal end of the main body 93; the distal end of the connector 91 has an inner structure and an outer structure, the inner structure is fixedly connected with the proximal end of the inner tube 2, and the outer structure is fixedly connected with the proximal end of the intermediate tube. The inner structure is fixedly connected with a terminal of a proximal end of the inner tube 2, and the outer structure is fixedly connected with a terminal of a proximal end of the intermediate tube 3.

When the operator pushes the sliding button 92, the sliding button 92 drives the connector 91, the intermediate tube 3, and the inner tube 2 to move, and the balloon 1 follows the intermediate tube 3 to move. Through the above structure, the operator is able to accurately control the movement of the balloon 1 through the sliding button 92.

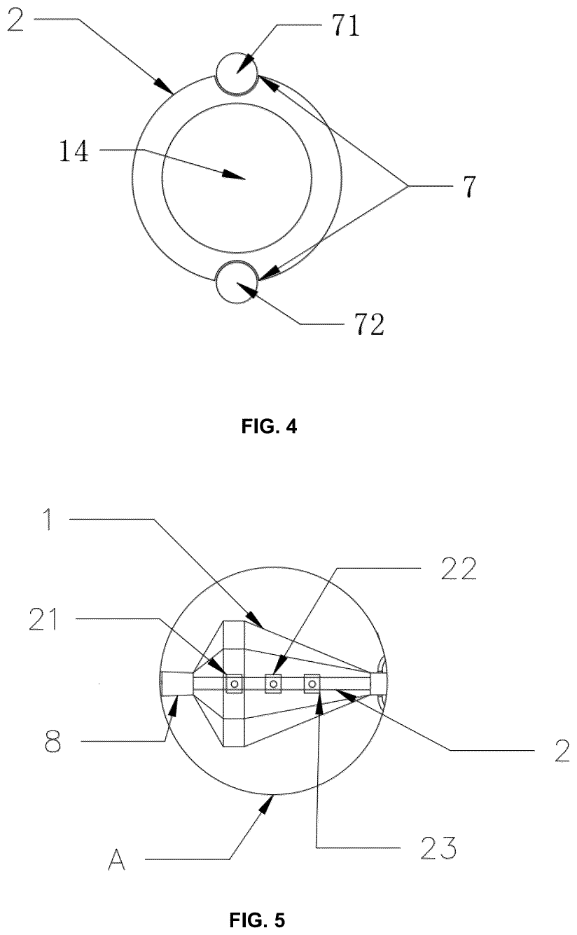

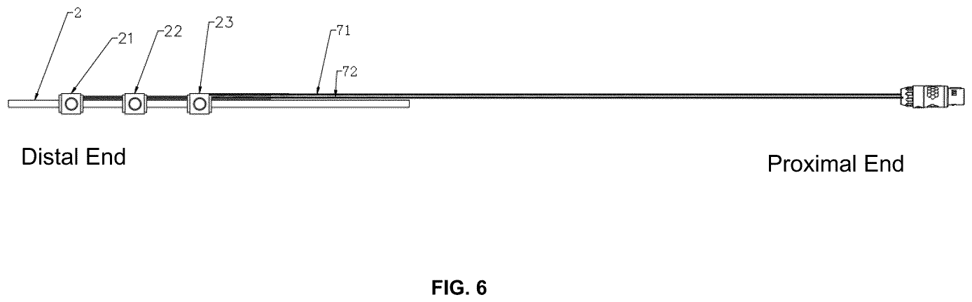

FIG. 4 is a schematic structural illustrating a cross-sectional structure of the inner tube according to some embodiments of the present disclosure. FIG. 5 is an enlarged view of region A in FIG. 1. FIG. 6 is a schematic diagram illustrating a structure of electrode assemblies according to some embodiments of the present disclosure.

In some embodiments, as shown in FIG. 4 to FIG. 6, one or more sets of electrode assemblies are provided at the inner tube 2 of the balloon structure 100, and each of the one or more sets of electrode assemblies is electrically connected with a positive electrode wire 71 and a negative electrode wire 72; two outer cavity channels are provided on an outer wall of the inner tube 2, and the positive electrode wire 71 and the negative electrode wire 72 are provided in the two outer cavity channels, respectively.

The one or more sets of electrode assemblies are configured to generate shockwaves by arcing through the fluid within the balloon. The shockwaves from the electrode assemblies are delivered to a surface of the balloon 1 via the fluid to apply shockwave therapy to the calcified valve. In some embodiments, a count of electrode assemblies is one set, and one set of electrode assemblies includes a positive electrode and a negative electrode. The positive electrode is connected with a positive terminal of the power supply via the positive electrode wire 71, and the negative electrode is connected with a negative terminal of the power supply via the negative electrode wire 72. As previously described, the positive electrode wire 71 and the negative electrode wire 72 may be connected with a high-voltage plug to provide a power source for the electrode assemblies.

In some embodiments, a count of the electrode assemblies is three set, the three sets of electrode assemblies are set up coaxially, and the three sets of electrode assemblies are connected in series. Each set of electrode assemblies includes an inner electrode, an insulating tube, and an outer electrode arranged in sequence from inside to outside, and a plurality of discharge holes is provided in the insulating tube and the outer electrode. In some embodiments, the three sets of electrode assemblies may also be connected in parallel. For example, inner electrodes of the three sets of electrode assemblies are connected together, and outer electrodes of the three sets of electrode assemblies are also connected together. In some embodiments, the inner electrode is the positive electrode and the outer electrode is the negative electrode. In other embodiments, the inner electrode is the negative electrode and the outer electrode is the positive electrode.

As shown in FIG. 5 and FIG. 6, the three sets of electrode assemblies include a first electrode set 21, a second electrode set 22, and a third electrode set 23, and axes of the three sets of electrode assemblies are the same as the axis of the inner tube 2.

In some embodiments, an inner electrode of the first electrode set 21 is connected with the negative electrode wire 72, an outer electrode of the first electrode set 21 is connected with an outer electrode of the second electrode set 22, an inner electrode of the second electrode set 22 is connected with an inner electrode of the third electrode set 23, and an outer electrode of the third electrode set 23 is connected with the positive electrode wire 71.

In some embodiments of the present disclosure, a connection of the positive and negative electrodes and electrode wires can be simplified by the connection manner of the electrodes and the electrode wires in the electrode assemblies.

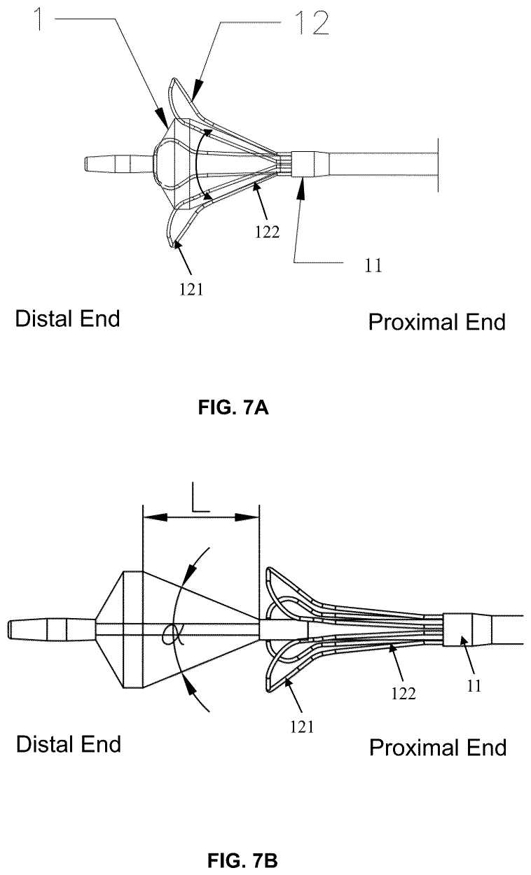

FIG. 7A is a schematic diagram illustrating a structure of a clamping arm device according to some embodiments of the present disclosure. FIG. 7B is a schematic diagram illustrating another structure of the clamping arm device according to some embodiments of the present disclosure.

In some embodiments, as shown in FIG. 7A and FIG. 7B, a clamping arm device 10 is fixedly arranged at a terminal of a distal end of the outer tube 4, and the clamping arm device 10 includes a metal seat 11 and a plurality of clamping arms 12 fixedly provided at a distal end of the metal seat 11.

The clamping arm device 10 refers to a device for further squeezing or clamping of the valve. The metal seat 11 may be provided in a position of a proximal end of the balloon 1. Merely by way of example, the metal seat 11 is a tubular structure, and an inner wall of an end portion of the metal seat 11 is fixedly connected with a terminal of the distal end of the outer tube 4; the clamping arm 12 is a structure of bent metal wire.

In some embodiments, the plurality of clamping arms 12 is made of nickel-titanium memory alloy material.

A shape of the clamping arm device 10, which utilizes the nickel-titanium memory alloy material, may be deformed to further squeeze or clamp the valve. For example, the shape of the plurality of clamping arms 12 of the clamping arm device 10 may be adapted to a shape of the vessel when not yet at the valve; when the plurality of clamping arms 12 reaches the position of the valve during the intervention, the angle of opening may be increased, thereby squeezing or clamping the valve. Merely by way of example, when the plurality of clamping arms 12 reach the valve position during the intervention and are fully deployed, the valve leaflets are positioned between the balloon 1 and the plurality of clamping arms 12, with the plurality of clamping arms 12 clamping the valve.

In some embodiments, the metal seat 11 has a hexagonal cross-section, the count of the plurality of clamping arms 12 is three, a head end 121 of each of the plurality of clamping arms 12 is in a flattened circular arc shape, and each of the plurality of the clamping arms includes two leg portions 122, each of the two leg portions 122 is fixedly connected with an apex of the metal seat 11. A diameter of the head end 121 of each of the plurality of clamping arms 12 is less than a diameter of each of the two leg portions 122.

Merely by way of example, the metal seat 11 has a hollow regular hexagonal cross-section; the six leg portions 122 of the three clamping arms 12 are fixedly connected to six vertices of the hexagon, respectively.

By designing the structure and dimensions of the clamping arm device 10, the balloon in conjunction with a specially shaped of the clamping arms, is capable of affixing an three valve leaflets to the balloon; and at the same time, the curvature of the balloon serves as a pivot point, and the two leg portions of the clamping arms exert a force in the direction of the pivot point, which is capable of increasing the magnitude of the force exerted by the shockwave balloon device on the calcified leaflets, thereby making it easier to fracture the calcified leaflets.

In some embodiments, an outer surface of each of the plurality of clamping arms 12 is coated or covered with a lubricating material. The lubricating material includes hydrophilic polymers, such as polyvinylpyrrolidone, polyacrylic acid, and hydrophobic polymers, such as polytetrafluoroethylene. The lubricating material can reduce friction and minimize frictional damage to the blood vessel caused by the clamping arms 12.

The structure of the three clamping arms, with the outer surfaces of the clamping arms coated or covered with the lubricating material, facilitates the reduction of damage to the valve by the shockwave balloon device and better clamping of the valve.

In some embodiments, the count of the plurality of clamping arms 12 is more, such as 4, 5, or 6; correspondingly, the metal seat 11 has a cross-section with 8 vertices, 10 vertices, or 12 vertices; and each of the two leg portions 122 is fixedly connected with a vertex of the metal seat 11. The shockwave balloon device with different counts of the clamping arms 12 is applicable in different scenarios. For example, a shockwave balloon device with four clamping arms 12 may be used at the mitral valve.

In some embodiments, a distance between the head end 121 of each of the plurality of clamping arms 12 and a distal end of the metal seat 11 ranges from 1 cm to 3 cm. A diameter of a fitted circle formed by a deployed tip opening of the head ends 121 of three of the plurality of clamping arms 12 ranges from 1.0 cm to 3.5 cm. For example, the distance between the head end 121 of each of the plurality of clamping arms 12 and the distal end of the metal seat 11 is 3 cm. The diameter of the fitted circle formed by the deployed tip opening of the head ends 121 of three of the plurality of clamping arms 12 is 3.5 cm. As another example, the distance between the head end 121 of each of the plurality of clamping arms 12 and the distal end of the metal seat 11 is 1 cm. The diameter of the fitted circle formed by the deployed tip opening of the head ends 121 of three of the plurality of clamping arms 12 is 2 cm. As a further example, the distance between the head end 121 of each of the plurality of clamping arms 12 and the distal end of the metal seat 11 is 2.5 cm. The diameter of the fitted circle formed by the deployed tip opening of the head ends 121 of three of the plurality of clamping arms 12 is 3.5 cm. As a further example, the distance between the head end 121 of each of the plurality of clamping arms 12 and the distal end of the metal seat 11 is 1 cm. The diameter of the fitted circle formed by the deployed tip opening of the head ends 121 of three of the plurality of clamping arms 12 is 1 cm.

In some embodiments, as shown in FIG. 2, FIG. 7A and FIG. 7B, the balloon 1 is of a circular-arc triangular-conical type, an included angle of a conical surface of the balloon 1 ranges from 28° to 60°, an axial length of a circular-arc surface of the balloon 1 is equal to an axial length of the conical surface of the balloon 1, and the axial length of the conical surface of the balloon 1 ranges from 1 cm to 5 cm. As shown in FIG. 7B, the axial length of the circular-arc surface of the balloon 1 and the axial length of the conical surface of the balloon 1 are both L. For example, the included angle of the conical surface of the balloon 1 is 55°, the axial length of the circular-arc surface of the balloon 1 is equal to the axial length of the conical surface of the balloon 1, the axial length of the conical surface of the balloon 1 is 3 cm, and the axial length of the circular-arc surface of the balloon 1 is also 3 cm. As another example, the included angle of the conical surface of the balloon 1 is 28°, the axial length of the circular-arc surface of the balloon 1 is equal to the axial length of the conical surface of the balloon 1, the axial length of the conical surface of the balloon 1 is 3 cm, and the axial length of the circular-arc surface of the balloon 1 is also 3 cm.

In some embodiments, a bending angle of each of the plurality of clamping arms 12 matches the included angle of the conical surface of the balloon, and an axial length of each of the plurality of clamping arms 12 matches an axial length of the conical surface of the balloon 1. For example, a taper of the profile of the clamping arm 12 when initially deployed is equal to or substantially equal to the included angle of the conical surface of the balloon 1.

For example, when the clamping arm 12 is initially deployed, the two leg portions 122 of the clamping arm 12 fit or are proximate to fit the included angle of the conical surface of the balloon 1. When the clamping arm 12 is initially deployed, a projection length of the clamping arm 12 from a proximal end to a distal end in an axial direction (i.e., an axial length of the clamping arm 12) is equal to or substantially equal to the axial length of the conical surface of the balloon 1.

In some embodiments of the present disclosure, a specially shaped of the balloon is utilized, with the balloon centered between the three valve leaflets, and the balloon causes the valve to be affixed to the balloon by cooperating with the specially shaped of the clamping arms; the balloon curvature serves as a fulcrum, and the two leg portions of the clamping arm apply force in the direction of the fulcrum, making it easier to fracture the calcified valve while clamping the valve; a center of the balloon, which is not in contact with the annulus, is able to reduce the effect of the shockwave energy on the electrical conduction of the heart. Three of the plurality of clamping arms clamp three valve leaflets, avoiding the impact of the heartbeat on the surgical process. The three of the plurality of clamping arms simultaneously clamp the three valve leaflets for shockwave therapy, which can increase the efficiency of the procedure.

In some embodiments, a distal end of the tip tube 8 has a chamfered opening with a tapered profile. The most distal end of the tip tube 8 forms a tip, which has a hardness of 30D.

Some embodiments of the present disclosure provide a method of using a shockwave balloon device, the method of use comprises: an operator carefully loading the shockwave device of the valve along a pre-implanted guidewire, slowly pushing a shockwave catheter (e.g., an outer tube 4), so that the outer tube 4 travels forward along an inner wall of the adjustable curved sheath until the outer tube reaches the upper part of the aortic valve. The interplay between the structural properties of the outer tube 4 during pushing reduces damage to the blood vessel wall. For example, the connecting section and the soft section of the outer tube 4 are set to the hardness of 35D, and the length of the soft section is 10 cm, which works in concert with the pushing section having a relatively high rigidity; the relatively high hardness of the pushing section facilitates pushing, and the moderate softness and specific length of the soft section effectively adapts to the curvature and softness of blood vessels.

The operator holds the sliding button 92 on the handle 9, which is fixedly connected with the proximal end of the intermediate tube 3, and the sliding button 92 drives the intermediate tube 3, thereby pushing the balloon 1. At the same time, the operator may utilize X-ray to closely observe the balloon 1 marking so as to push the balloon 1 underneath the valve, at which time the three of the plurality of clamping arms 12 of the clamping arm device 10 at the distal end of the outer tube 4 can be fully released.

The operator then adjusts the X-ray angle to observe the position of the three of the plurality of clamping arms and the movement of the valve; if deviations in the position of the clamping arms are detected, the position of the clamping arms can be finely adjusted by gently rotating the catheter until the clamping arms are perfectly aligned with the three sinus positions of the aorta, ensuring that the clamping arms acts uniformly and stably on the valve. The operator may also use the ultrasound device to observe the motion of the valve, further confirming the details of the valve and the relative position of the device to the valve.

Filling the balloon 1 with the contrast agent through the first channel 5 can provide an efficient path for filling the balloon 1 and circulating the internal fluid. When the balloon 1 is pressurized to the prescribed pressure, under real-time observation by ultrasound, the operator may manipulate the sliding button 92 on the handle 9 to move the balloon 1, so that the valve is tightly clamped by the three clamping arms 12.

After the operator presses the treatment button on the connector cable of the Intravascular Lithotripsy (IVL) treatment system, the treatment system delivers 10 pulses over a preset period of time. At this time, the three sets of electrode assemblies in the inner tube 2 begin to work, and the three sets of electrode assemblies generate shock waves by arcing through the liquid in the balloon, and the shock waves are delivered to the surface of the balloon 1 via the liquid, thus implementing shock wave therapy for the calcified valve; after completing the delivery of the pulse, the operator retracts the balloon 1 completely and carefully withdraw the adjustable curved sheath and guidewire, thus completing the entire surgical procedure.

In some embodiments of the present disclosure, by setting up a specially shaped of the balloon, which is centered between the three valve leaflets, and the balloon is made to affix the valve to the balloon by cooperating with a specially shaped of the plurality of clamping arms, a curvature of the balloon serves as a fulcrum, and the two leg portions of the plurality of clamping arms exert force in the direction of the fulcrum, making it easier to fracture the calcified valve while clamping the valve. The center of the balloon does not contact the annulus, which reduces the impact of shock wave energy on cardiac conduction, and the three clamping arms clamp the three valve leaflets, which avoids the impact of the heartbeat on the surgical procedure. The three clamping arms simultaneously clamp the three valve leaflets for shockwave therapy, which can increase the efficiency of the procedure, and the device is set up with a contrast agent channel above the valve and below the valve during the treatment, which does not require additional contrast agent catheter intervention, thus reducing trauma to the patient and increasing visualization for the surgeon.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or collocation of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer-readable program code embodied thereon.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required characteristics of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. A shockwave balloon device, comprising a handle structure and a balloon structure connected with the handle structure; wherein
the handle structure includes a handle and an outer tube, the outer tube is fixedly disposed at a distal end of the handle, the handle is slidably disposed with a sliding button, and the sliding button is fixedly connected with a proximal end of the balloon structure;
the balloon structure includes an intermediate tube, an inner tube, a balloon, and a tip tube, the intermediate tube is sheathed on an outer side of the inner tube, the balloon is sheathed on the outer side of the inner tube, a proximal end of the intermediate tube is fixedly connected with the sliding button, a distal end of the intermediate tube is fixedly connected with the balloon, and the tip tube is fixedly disposed at a distal end of the balloon; and
the intermediate tube is slidably coupled with the outer tube.

2. The shockwave balloon device according to claim 1, wherein
one or more sets of electrode assemblies are provided at the inner tube of the balloon structure, and each of the one or more sets of electrode assemblies is electrically connected with a positive electrode wire and a negative electrode wire, two outer cavity channels are provided on an outer wall of the inner tube, and the positive electrode wire and the negative electrode wire are provided in the two outer cavity channels, respectively.

3. The shockwave balloon device according to claim 1, wherein
a first gap is arranged between the inner tube and the intermediate tube to form a first channel, the first channel is in communication with a cavity within the balloon; and
a second gap is arranged between the intermediate tube and the outer tube to form a second channel.

4. The shockwave balloon device according to claim 3, wherein the handle includes a main body and a connector;
the connector includes a third channel, a fourth channel, and a fifth channel; wherein,
the third channel, the fourth channel, and the fifth channel penetrate the main body, the third channel is in communication with a tube lumen of the inner tube, the fourth channel is provided for passage of the positive electrode wire and the negative electrode wire, and the fifth channel is in communication with the first channel.

5. The shockwave balloon device according to claim 1, wherein
a clamping arm device is fixedly arranged at a distal end of the outer tube, the clamping arm device includes a metal seat, and a plurality of clamping arms is fixedly provided at a distal end of the metal seat.

6. The shockwave balloon device according to claim 5, wherein the plurality of clamping arms are made of nickel-titanium memory alloy material.

7. The shockwave balloon device according to claim 5, wherein
the metal seat has a hexagonal cross-section, the count of the plurality of clamping arms is three, a head end of each of the plurality of clamping arms is in a flattened circular arc shape, each of the plurality of clamping arms includes two leg portions, each of the two leg portions is fixedly connected with an apex of the metal seat, a diameter of the head end of each of the plurality of clamping arms is less than a diameter of each of the two leg portions, and an outer surface of each of the plurality of clamping arms is coated or covered with a lubricating material.

8. The shockwave balloon device according to claim 7, wherein
a distance between the head end of each of the plurality of clamping arms and a distal end of the metal seat ranges from 1 cm to 3 cm, and a diameter of a fitted circle formed by a deployed tip opening of the head ends of three of the plurality of clamping arms ranges from 1.0 cm to 3.5 cm.

9. The shockwave balloon device according to claim 7, wherein the balloon is of a circular-arc triangular-conical type, an included angle of a conical surface of the balloon ranges from 28° to 60°, an axial length of a circular-arc surface of the balloon is equal to an axial length of the conical surface of the balloon, and the axial length of the conical surface of the balloon ranges from 1 cm to 5 cm.

10. The shockwave balloon device according to claim 7, wherein a bending angle of each of the plurality of clamping arms matches the included angle of the conical surface of the balloon, and an axial length of each of the plurality of clamping arms matches an axial length of the conical surface of the balloon.

11. The shockwave balloon device according to claim 1, wherein a hemostatic valve is fixedly provided at a proximal end of the outer tube; and
the handle includes a main body; wherein an interior of the main body has a cavity and the hemostatic valve is fixedly disposed in the main body.

12. The shockwave balloon device according to claim 11, wherein the hemostatic valve includes a first outlet channel and a second outlet channel;
a silicone sealing ring is fixedly provided in the first outlet channel, the proximal end of the intermediate tube passes through the silicone sealing ring, and the silicone sealing ring is configured to seal the second channel; and
the second outlet channel is in communication with the second channel, a side of the second outlet channel away from the outer tube extends beyond a side wall of the main body, and the second outlet channel is fixedly connected with a three-way valve.

13. The shockwave balloon device according to claim 12, wherein a compressive strength range of the silicone sealing ring is 200 psi to 500 psi.

14. The shockwave balloon device according to claim 12, wherein
the intermediate tube includes a distal section and a proximal section, the distal section is made of a braided tube constructed from interwoven filaments, a hardness of the distal section is in a range of 50D~70D, the proximal section of the intermediate tube is a metal tube, and a length of the proximal section is greater than a distance from the connector to the silicone sealing ring at the three-way valve.

15. The shockwave balloon device according to claim 1, wherein the outer tube is a connecting section, a soft section, and a pushing section in order from the distal end to the proximal end.

16. The shockwave balloon device according to claim 15, wherein the soft section and the pushing section are made of braided filaments, a hardness of the pushing section is greater than a hardness of the soft section, the hardness of the soft section is in a range of 35D~55D, and a length of the soft section is in a range of 10cm~35cm; and
an inner wall of the outer tube is coated with a PTFE coating or lubricant.

17. The shockwave balloon device according to claim 1, wherein
the handle includes a main body and a connector; a proximal end of the connector penetrates a proximal end of the main body, the connector is slidably disposed at the proximal end of the main body, a groove is provided in a side of the main body, the sliding button is slidably disposed in the groove, and a side of the sliding button is fixedly connected with an outer wall of a proximal end of the intermediate tube; and
the proximal end of the intermediate tube and a proximal end of the inner tube are fixedly connected with a distal end of the connector.

18. The shockwave balloon device according to claim 2, wherein a count of the one or more sets of electrode assemblies is three, the three sets of electrode assemblies are set up coaxially, and the three sets of electrode assemblies are connected in series; and
each set of electrode assemblies includes an inner electrode, an insulating tube, and an outer electrode arranged in sequence from inside to outside, and a plurality of discharge holes are provided in the insulating tube and the outer electrode.

19. The shockwave balloon device according to claim 18, wherein
an inner electrode of a first electrode set is connected with the negative electrode wire, an outer electrode of the first electrode set is connected with an outer electrode of a second electrode set, an inner electrode of the second electrode set is connected with an inner electrode of a third electrode set, and an outer electrode of the third electrode set is connected with the positive electrode wire.
